(19) United States
(12) Patent Application Publication
(10) Pub. No.: US 2002/0137660 A1
(43) Pub. Date: Sep. 26, 2002

(54) Process for the preparation of alkylcarboxylic allyl esters

(76) Inventors: Walter Kuhn, Holzminden (DE); Gerhard Senft, Holzminden (DE)

Correspondence Address: BAYER CORPORATION, PATENT DEPARTMENT, 100 BAYER ROAD, PITTSBURGH, PA, 15205, US

(21) Appl. No.: 10/100,344

(22) Filed: Mar. 18, 2002

(30) Foreign Application Priority Data
Mar. 22, 2001 (DE) 10113964.0

(51) Int. Cl. A61K 7/46; C07C 69/00; C07C 61/08; C07C 51/14

(52) U.S. Cl. 512/8; 512/22; 512/25; 512/26; 560/129; 562/400; 562/517; 562/519

(57) ABSTRACT

The present invention relates to a process for the preparation of alkylcarboxylic allyl esters by reacting allyl alcohol and alkylcarboxylic acids.

### FIELD OF THE INVENTION

The present invention relates to a process for the preparation of alkylcarboxylic allyl esters by reacting allyl alcohol and alkylcarboxylic acids.

### BACKGROUND OF THE INVENTION

Alkylcarboxylic allyl esters are sought-after odorants. S. Arctander, Perfume and Flavour Chemicals, No. 70, 71, 85, 77, 88; 1969 describes the esters allyl hexanoate, allyl heptanoate, allyl octanoate, allyl cyclohexylpropionate and allyl nonanoate. These esters are characterized by their fruity, pineapple-like character. The direct route to alkylcarboxylic allyl esters is given by the azeotropic, acid-catalyzed esterification of allyl alcohol (2-propen-1-ol) and the corresponding alkylcarboxylic acid with the assistance of a water entrainer. The water entrainers customarily used are solvents which form azeotropes with water, such as, for example, benzene, toluene, hexane or cyclohexane.

Journal of Perfumer and Flavorist, Vol. 25, January-February 2000, p. 20 describes the reaction of allyl alcohol and cyclohexylpropionic acid to give allyl cyclohexyl propionate using n-hexane as a water entrainer and para-toluenesulfonic acid as a catalyst. Allyl alcohol is used here in a 5-fold excess.

The use of said azeotrope-forming solvents is undesired. It may also have a negative effect on the sensory impression. Increased purification expenditure is, therefore, necessary to separate off the water entrainer.

### SUMMARY OF THE INVENTION

Therefore, it is an object of the present invention to find a process for the preparation of alkylcarboxylic allyl esters which permits a simple route to sensorily perfect alkylcarboxylic allyl esters.

The present invention relates to a process for the preparation of alkylcarboxylic allyl esters comprising the reacting of an alkylcarboxylic acid with allyl alcohol in the presence of a catalyst acid, wherein an alkylcarboxylic allyl ester is added at the start of the reaction.

### DETAILED DESCRIPTION OF THE INVENTION

A preferred embodiment is that no additional solvent functioning as a water entrainer is added. A preferred embodiment is the alkylcarboxylic allyl ester to be formed is added to the mixture of alkylcarboxylic acid and catalyst acid prior to the start of the reaction.

The present invention further provides for a process for the preparation of alkylcarboxylic allyl esters by reacting an alkylcarboxylic acid with allyl alcohol in the presence of a catalyst acid, wherein no additional solvent functioning as a water entrainer is used.

Diallyl ether can likewise be added prior to the start of the reaction. Diallyl ether can form as a by-product in the esterification reaction and may be present in the azeotrope which distills off.

The alkylcarboxylic allyl esters prepared according to the processes are primarily used as odorants, in perfume compositions, perfume oils or scent compositions.

Preferred alkylcarboxylic acids suitable according to the present invention correspond to the formula R¹—COOH, where R¹ may be straight-chain, branched or cyclic alkyl radicals. Preference is given to alkyl radicals R¹ containing 3 to 12 carbon atoms. An example of a cycloalkylcarboxylic acid which may be mentioned is 3-cyclohexylpropionic acid.

Examples of R¹ which may be mentioned are: n-propyl, iso-propyl, n-butyl, 2-butyl, iso-butyl, n-pentyl, iso-pentyl, 2-pentyl, 3-pentyl, n-hexyl, neo-hexyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 3,4-dimethylpentyl, 3,3-dimethylpentyl, 4,4-dimethylpentyl, n-heptyl, iso-heptyl, n-octyl, iso-octyl, n-nonyl, 2-ethylhexyl, 2,3-diethylpentyl, 2,4-diethylpentyl, 3,4-diethylpentyl, 2-ethylheptyl, 3-ethylheptyl, 4-ethylheptyl, 2-ethyloctyl, 3-ethyloctyl, 4-ethyloctyl, 5-ethyloctyl, 3-ethyl-2-methylhexyl, 2-ethyl-3-methylhexyl, 2-ethyl-5-methylhexyl, 3-ethyl-5-methylhexyl, cyclopropyl, cyclobutyl, cyclopentyl, 2-methylcyclopentyl, 3-methylcyclopentyl, 4-methylcyclopentyl, 2,3-dimethylcyclopentyl, 2,4-dimethylcyclopentyl, 3,3-dimethylcyclopentyl, 4,4-dimethylcyclopentyl, 2,3-diethylcyclopentyl, 2,4-diethylcyclopentyl, 2-ethyl-3-methylcyclopentyl, 3-ethyl-2-methylcyclopentyl, 3-ethyl-4-methylcyclopentyl, 4-ethyl-3-methylcyclopentyl, cyclohexyl, 2-methylcyclohexyl, 3-methylcyclohexyl, 4-methylcyclohexyl, 2-ethylcyclohexyl, 3-ethylcyclohexyl, 4-ethylcyclohexyl, 5-ethylcyclohexyl, 2-cyclohexylethyl, 3-cyclohexylpropyl, 3-cyclohexylbutyl, 4-cyclohexylbutyl, cyloheptyl, cylononyl, cyclodecyl and cyclododecyl.

According to the present invention, preference is given to using pentanoic acid, hexanoic acid, heptanoic acid, octanoic acid, cyclohexylpropionic acid or nonanoic acid as the alkylcarboxylic acid.

More preference is given to n-pentanoic acid, n-hexanoic acid, n-heptanoic acid, n-octanoic acid, 3-cyclohexylpropionic acid or n-nonanoic acid.

Suitable catalyst acids are strong acids, such as sulfuric acid, phosphoric acid, p-toluenesulfonic acid or hydrochloric acid.

The reaction is carried out according to the present invention at 70-170° C., preferably at 120-140° C.

The process according to the present invention can be carried out within a wide pressure range, and preference is given to carrying out the reaction at atmospheric pressure.

The quantitative data and ratios below refer to molar amounts and data.

The suitable use amount of catalyst acid based on the use amount of alkylcarboxylic acid is in the range 0.0001 to 0.05:1, preferably in the range 0.001 to 0.01:1.

The ratio of allyl alcohol based on the alkylcarboxylic acid can be within wide ranges from 0.1:1 to 10:1, suitably in the range from 0.5:1 to 5:1. Preference is given to using allyl alcohol in an excess in the range 1 to 2:1, preferably in the range 1.1 to 1.5:1.

If the reaction of the alkylcarboxylic acid with allyl alcohol is carried out in the absence of an alkylcarboxylic allyl ester, then the reaction proceeds somewhat more slowly under identical reaction conditions, and the content of alkylcarboxylic allyl ester after the reaction is in most cases lower than in cases where an alkylcarboxylic allyl ester is added.

If an alkylcarboxylic allyl ester is cointroduced into the reaction, then this can take place within wide ranges. Based on the molar amount of alkylcarboxylic acid used, the added alkylcarboxylic allyl ester can be added in the ratio 0.001 to 0.8:1, preferably in the ratio 0.01 to 0.03:1.

In a preferred embodiment, the alkylcarboxylic allyl ester to be formed is added, preferably prior to the start of the reaction.

According to the present invention, preferred alkylcarboxylic allyl esters are allyl pentanoate, allyl hexanoate, allyl heptanoate, allyl octanoate, allyl cyclohexylpropionate or allyl nonanoate.

More preference is given to allyl n-pentanoate, allyl n-hexanoate, allyl n-heptanoate, allyl n-octanoate, allyl cyclohexylpropionate or allyl n-nonanoate.

The process according to the present invention can, for example, be carried out as follows:

Allyl alcohol is metered into the initial charge of alkylcarboxylic acid R¹—COOH and catalyst acid or a mixture of alkylcarboxylic acid R¹—COOH, catalyst acid and optionally an alkylcarboxylic allyl ester, preferably the alkylcarboxylic allyl ester R¹—COO—CH₂—CH═CH₂ to be formed. At the same time, the distillate, which contains water, allyl alcohol and alkylcarboxylic allyl ester, is passed via a water separator. In so doing, water is continuously separated off. Where appropriate, a further amount of allyl alcohol can be added to the reaction mixture in order to complete the conversion of the reaction. After the end of the reaction, washing operations can follow and the product can be obtained by simple distillation.

The examples below illustrate the invention:

### EXAMPLES

### Example 1

Preparation of allyl n-hexanoate

2340 g (20.2 mol) of n-hexanoic acid, 50 g (0.3 mol) of allyl n-hexanoate and 19 g (0.1 mol) of p-toluenesulphonic acid are initially introduced at 140° C. 1150 g (19.8 mol) of allyl alcohol are metered in at 130-140° C. over 7 hours. At the same time, the azeotrope is distilled off via a water separator. 480 g of aqueous phase are separated off. At the end of the water separation, a further 150 g (2.6 mol) of allyl alcohol are metered in at 130-140° C. over 1 hour, and 319 g of azeotrope are distilled off simultaneously. Towards the end of the metered addition, distillation is started under reduced pressure at 300 mbar and at 120° C. After the reaction mixture has been cooled, it is washed with 200 g of water and then with 600 g of 5% strength NaOH solution.

After simple distillation at 78° C./32 mbar, 3,298 g of crude product give 3075 g of allyl n-hexanoate with a purity of >99%.

The theoretical yield of allyl n-hexanoate based on the use of n-hexanoic acid is 96%.

### Example 2

Preparation of allyl n-heptanoate

2,380 g (18.3 mol) of n-heptanoic acid, 50 g (0.3 mol) of allyl n-heptanoate and 19 g (0.1 mol) of p-toluenesulfonic acid are initially introduced at 140° C. 1,150 g (19.8 mol) of allyl alcohol are metered in at 130-140° C. over 7 hours. At the same time, the azeotrope is distilled off via a water separator. 438 g of aqueous phase are separated off. When water separation is complete, a further 150 g of allyl alcohol (2.6 mol) are metered in at 130-140° C. over 1 hour. Towards the end of the metered addition, distillation is started at reduced pressure at 300 mbar and at 120° C. 273 g of azeotrope distill over.

After the reaction mixture has been cooled, it is washed with 200 g of water and then with 600 g of 5% strength NaOH solution. After simple distillation at 91° C./21 mbar, 3,182 g of crude product give 2,943 g of allyl n-heptanoate with a purity of >99%.

The theoretical yield of allyl n-heptanoate based on the use of n-heptanoic acid is 93%.

### Example 3

Preparation of allyl n-hexanoate without the addition of alkylcarboxylic allyl ester

A reaction was carried out as described in Example 1 but without the addition of allyl n-hexanoate. The resulting crude product comprised 86% of the desired allyl n-hexanoate prior to distillation.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process for the preparation of alkylcarboxylic allyl esters comprising the step of reacting an alkylcarboxylic acid with allyl alcohol in the presence of a catalyst acid, wherein an alkylcarboxylic allyl ester is added at the start of the reaction.

2. A process according to claim 1, wherein no additional solvent functioning as a water entrainer is added.

3. A process according to claim 1, wherein an alkylcarboxylic acid R¹—COOH is used, where the alkyl radical R¹ contains 3 to 12 carbon atoms and may be straight-chain, branched or cyclic.

4. A process according to claim 1, wherein said alkylcarboxylic acid is selected from the group consisting of n-pentanoic acid, n-hexanoic acid, n-heptanoic acid, n-octanoic acid, 3-cyclohexylpropionic acid or n-nonanoic acid.

5. A process according to claim 1, wherein the molar use amount of allyl alcohol based on alkylcarboxylic acid is in the range 0.1:1 to 10:1.

6. A process according to claim 5, wherein the molar use amount of allyl alcohol based on alkylcarboxylic acid is in the range 1:1 to 2:1.

7. A process according to claim 1, wherein an amount of the alkylcarboxylic allyl ester to be formed is added to the mixture of alkylcarboxylic acid and catalyst acid prior to the start of the reaction.

8. A process according to claim 1, wherein said alkylcarboxylic allyl ester is selected from the group consisting of allyl n-pentanoate, allyl n-hexanoate, allyl n-heptanoate, allyl n-octanoate, allyl 3-cyclohexylpropionate or allyl n-nonanoate.

9. A process according to claim 1, wherein the molar use amount of added alkylcarboxylic allyl ester based on alkylcarboxylic acid is in the range 0.001 to 0.8:1.

10. A process according to at least claim 9, wherein the molar use amount of the added alkylcarboxylic allyl ester based on alkylcarboxylic acid is in the range 0.01 to 0.3:1.

11. A process according to claim 1, wherein the reaction is carried out at a temperature in the range 70-170° C.

12. A process according to claim 11, wherein the reaction is carried out at a temperature in the range 120-140° C.

13. A process according to claim 1, wherein diallyl ether is added to the mixture of alkylcarboxylic acid and catalyst acid prior to the start of the reaction.

14. A process for the preparation of alkylcarboxylic allyl esters by reacting an alkylcarboxylic acid with allyl alcohol in the presence of a catalyst acid, wherein no additional solvent functioning as water entrainer is used.

15. A process according to claim 14, wherein the alkylcarboxylic acid is selected from the group consisting of n-pentanoic acid, n-hexanoic acid, n-heptanoic acid, n-octanoic acid, 3-cyclohexylpropionic acid and n-nonanoic acid.

16. A process according to claim 14, wherein the molar use amount of allyl alcohol based on alkylcarboxylic acid is in the range 0.1:1 to 10:1.

17. A process according to claim 14, wherein the reaction is carried out at a temperature in the range 70-170° C.

18. Odorants comprising alkylcarboxylic allyl esters prepared by reacting an alkylcarboxylic acid with allyl alcohol in the presence of a catalyst acid, wherein an alkylcarboxylic allyl ester is added at the start of the reaction.

19. Perfume compositions, perfume oils or scent compositions comprising alkylcarboxylic allyl esters which have been prepared by reacting an alkylcarboxylic acid with allyl alcohol in the presence of a catalyst acid, wherein an alkylcarboxylic allyl ester is added at the start of the reaction.
